(19) United States
(12) Patent Application Publication
(10) Pub. No.: US 2026/0357208 A1
(43) Pub. Date: Nov. 12, 2026

(54) METHOD FOR DETECTION OF ANTIBODY-DEPENDENT CELLULAR PHAGOCYTOSIS

(71) Applicant: JANSSEN PHARMACEUTICA NV

(72) Inventors: Chi Kwong SO, Horsham, PA (US); Tammy SODEN, Horsham, PA (US); Wen-I TSOU, Raritan, NJ (US); Diana STAFA, Raritan, NJ (US); Wenyu LI, Raritan, NJ (US)

(21) Appl. No.: 19/120,888

(22) PCT Filed: Oct. 13, 2023
(86) PCT No.: PCT/IB2023/060349

§ 371 Date: Apr. 14, 2025

Related U.S. Application Data
(60) Provisional application No. 63/379,599, filed on Oct. 14, 2022.
(60) Provisional application No. 63/379,612, filed on Oct. 14, 2022.

(51) Int. Cl. C12N 5/0786; G01N 33/50; G01N 33/58

(52) U.S. Cl. CPC C12N 5/0645; G01N 33/5005; G01N 33/582; C12N 2500/02

(57) ABSTRACT

Provided is a method for increasing antibody-dependent cellular phagocytosis (ADCP) activity of a phagocytic cell in a sample comprising: culturing the phagocytic cell in a low percent (%) CO₂ atmosphere for a period of time sufficient to increase ADCP activity of the phagocytic cell. In some embodiments, the phagocytic cells are cultured in the presence of target cells. Also provided is a method for assaying ADCP activity of a phagocytic cell in a sample comprising: culturing the phagocytic cell in a low percent (%) CO₂ atmosphere for a period of time sufficient to increase ADCP activity of the phagocytic cell; and detecting ADCP activity of the phagocytic cell in the sample. In some embodiments, the phagocytic cells are cultured in the presence of target cells.

### CROSS REFERENCE TO RELATED APPLICATIONS

This application is entitled to priority under 35 U.S.C. § 119(e) to U.S. Provisional Application Nos. 63/379,599, filed Oct. 14, 2022, and 63/379,612, filed Oct. 14, 2022, each of which is hereby incorporated by reference in its entirety.

### FIELD

The general inventive concepts relate to the field of methods of detecting antibody-dependent cellular phagocytosis (ADCP) that are useful for quantifying the ADCP activity of antibodies, and in particular for quantifying the ADCP activity of therapeutic antibodies useful for the treatment of cancer.

### BACKGROUND

A number of therapeutic antibodies have been developed and approved for use in the treatment of cancer (Mossner et al., 2010; Shuptrine, Surana, & Weiner, 2012; Weiner, Murray, & Shuptrine, 2012; Weiner, Surana, & Wang, 2010). These therapeutic antibodies can affect tumor growth directly by interfering with receptor signaling, by blocking receptor downstream signaling, and by inducing apoptosis, but they can also affect tumor growth indirectly by activating complement-dependent cytotoxicity and affecting immune cell effector functions such as antibody-dependent cellular cytotoxicity (ADCC) and antibody-dependent cellular phagocytosis (ADCP) (Herter et al., 2014; Weiner et al., 2010). Unlike ADCC, which depends on effector cells to secrete molecules that activate apoptosis-based cell death, ADCP depends on macrophages to directly destroy target cells by phagocytosis. These macrophages in particular are poised to be tremendous effectors of cancer immunotherapy.

Macrophages reside in tissues throughout the body (Geissmann et al., 2010) and specialized tissue-specific macrophage populations exist, e.g., Kupffer cells in the liver, microglia in the brain, osteoclasts in bone, and alveolar macrophages in the lungs. And importantly, macrophages are known to infiltrate solid tumors thus gaining proximity to the tumor cells. These macrophages are crucial to the efficacy of many antibodies. In particular, in vivo and in vitro studies have indicated that ADCP is an important and potent mechanism of action (MOA) for the treatment of different cancers with different therapeutic antibodies, e.g., treatment of multiple myeloma (MM) with Daratumumab, an anti-CD38 antibody (Khagi & Mark, 2014; Overdijk et al., 2015), and treatment of B-cell malignancies with Rituximab, an anti-CD20 antibody (Oflazoglu & Audoly, 2010), have both been shown to have an important ADCP component. ADCP of solid tumors has also been demonstrated in vitro using anti-HER-2/neu antibodies with breast cancer cells (Watanabe et al., 1999) and using anti-epidermal growth factor receptor (EGFR) antibodies with colon cancer cells (Weiskopf et al., 2013). In addition, comparability studies have shown the importance of differences in ADCP for different therapeutic antibodies that bind the same target, e.g., anti-CD20 antibodies Ofatumumab, Obinutuzumab, and Rituximab (Rafiq et al., 2013).

The process of ADCP is essentially a two-step mechanism involving (1) binding of an antibody to a target cell, e.g., a tumor cell or a suitable target cell mimicking a patient's tumor; and (2) macrophages or other phagocytic cells such as monocytes, neutrophils or dendritic cells binding to the fragment crystallizable (Fc) domain of the antibody through Fc receptors on the phagocytic cells with subsequent phagocytosis of the target cell. These Fc receptors that mediate ADCP on the phagocytic cells include FcγRIIa (CD32a), FcγRI (CD64) and FcγRIIIa (CD16a), however, a number of studies suggest that FcγRIIa is the predominant FcγR receptor involved in induction of ADCP by macrophages (Richards et al., 2008; Weiskopf & Weissman, 2015).

In response to the recognition and appreciation of the role of ADCP as an important MOA in effective cancer treatments, regulatory authorities are now requiring data on the impact of ADCP on antibody mediated cytotoxicity for approval of therapeutic antibodies. In addition, ADCP assays can also provide insight into the stability of therapeutic antibodies at different stages of production or storage of the therapeutic product. As a result, drug developers and researchers are rapidly adopting ADCP assays as a key step during development and production of the therapeutic antibodies. Unfortunately, however, the prototypical ADCP assay using primary macrophages derived from human donors and target cell phagocytosis as an end-point can be a very challenging and time-consuming with variability that makes it unsuitable as a routine assay. In particular, there is long differentiation time for the primary macrophages generated in vitro from the peripheral blood mononuclear cell (PBMC) preparations derived from human donors and donor-to-donor differences can add considerable variability to the assays. Furthermore, the signal to noise ratio can be less than optimal for distinguishing ADCP activity from non-specific background.

### SUMMARY

Provided is a method for increasing antibody-dependent cellular phagocytosis (ADCP) activity of a phagocytic cell in a sample comprising: culturing the phagocytic cell in a low percent (%) CO₂ atmosphere for a period of time sufficient to increase ADCP activity of the phagocytic cell.

In some embodiments, the low % CO₂ is from about 0.1% CO₂ to about 1% CO₂. In some embodiments, the low % CO₂ is 0.1, 0.2, 0.3, 0.4, 0.5, 0.6, 0.7, 0.8, 0.9, or 1% CO₂. In further embodiments, the low % CO₂ is about 1% CO₂.

In some embodiments, the period of time sufficient to increase ADCP activity of the phagocytic cells is 3 to 15 days. In further embodiments, the period of time sufficient to increase ADCP activity of the phagocytic cell is 3 to 7 days.

In some embodiments, the method further comprises determining the ADCP activity of the phagocytic cell using target cells labeled with a fluorescent dye.

In some embodiments, the phagocytic cell is a J774A.1 cell or a donor cell. In further embodiments, the donor cell is a human macrophage cell.

In some embodiments, the phagocytic cells are cultured in the presence of target cells. In further embodiments, target cells are added to the phagocytic cell culture after about 1 to about 14 days. In yet further embodiments, target cells are added to the phagocytic cell culture after about 1, 2, 3, 4, 5, 6, 7, 8, 9, 10, 11, 12, 13, or 14 days. In certain embodiments, target cells are added to the phagocytic cell culture after about 7 days. In further embodiments, the target cells are Daudi cells, B cells, leukemia cells or lymphoma cells. In yet further embodiments, the target cells are Daudi cells.

In some embodiments, the fluorescent dye is pHrodo-Red, pHAb or AcidiFluor.

In some embodiments, expression of a regulator of ADCP is increased. In further embodiments, the regulator is ATF4, FOXO3, IL1B, IL6, VEGFA, HGF, EGF, CHD1, SELP, TIMP3, DACH1, STAT3, GLI1, SP3, or combinations thereof.

In some embodiments, expression of a regulator of ADCP is decreased. In further embodiments, the regulator is TP53, TNF, TGFB1, STAT6, MYD88, HRAS or combinations thereof.

In some embodiments, the target cells are contacted with an antibody or fragment thereof.

In some embodiments, the sample is cultured in a humidified chamber or incubator.

Provided is a method for assaying ADCP activity of a phagocytic cell in a sample comprising: culturing the phagocytic cell in a low percent (%) CO₂ atmosphere for a period of time sufficient to increase ADCP activity of the phagocytic cell; and detecting ADCP activity of the phagocytic cell in the sample.

In some embodiments, the low % CO₂ is from about 0.1% CO₂ to about 1% CO₂. In some embodiments, the low % CO₂ is 0.1, 0.2, 0.3, 0.4, 0.5, 0.6, 0.7, 0.8, 0.9, or 1% CO₂.

In some embodiments, the low % CO₂ is 1% CO₂.

In some embodiments, the period of time sufficient to increase ADCP activity of the phagocytic cells is 3 to 15 days. In further embodiments, the period of time sufficient to increase ADCP activity of the phagocytic cell is 3 to 7 days.

In some embodiments, the method further comprises determining the ADCP activity of the phacobytic cell using target cells labeled with a fluorescent dye.

In some embodiments, the phagocytic cell is a J774A.1 cell or a donor cell. In further embodiments, the donor cell is a human macrophage cell.

In some embodiments, the phagocytic cells are cultured in the presence of target cells. In further embodiments, target cells are added to the phagocytic cell culture after about 3 to about 10 days, for example after about 7 days.

In some embodiments, the target cells are labeled with a fluorescent dye.

In some embodiments, the phagocytic cell is a J 774A.1 cell or a donor cell. In further embodiments, the donor cell is a human macrophage cell.

In some embodiments, the target cells are Daudi cells, B cells, leukemia cells or lymphoma cells. In yet further embodiments, the target cells are Daudi cells.

In some embodiments, the fluorescent dye is pHrodo-Red, pHAb or AcidiFluor.

In some embodiments, expression of a regulator of ADCP is increased. In further embodiments, the regulator is ATF4, FOXO3, IL1B, IL6, VEGFA, HGF, EGF, CHD1, SELP, TIMP3, DACH1, STAT3, GLI1, SP3, or combinations thereof.

In some embodiments, expression of a regulator of ADCP is decreased. In further embodiments, the regulator is TP53, TNF, TGFB1, STAT6, MYD88, HRAS or combinations thereof.

In some embodiments, the target cells are contacted with an antibody or fragment thereof.

In some embodiments, the sample is cultured in a humidified chamber or incubator.

### DESCRIPTION OF THE FIGURES

FIGS. 1A-1B illustrate ADCP assay assessed by flow cytometry with human donor macrophages cultured in 5% CO₂. FIG. 1A: Representative flow cytometry quadrats data. FIG. 1B: Dose response curves (0.03 to 2000 ng/ml) for Daratumumab, filled circle (●), or Rituximab, filled triangle (▴).

FIGS. 2A-2B illustrate ADCP assay assessed by flow cytometry with J774A.1 cells cultured in 5% CO₂. FIG. 2A: Representative flow cytometry quadrats data. FIG. 2B: Dose response curve (0.03 to 2000 ng/ml) for Daratumumab, filled circle (●); or Rituximab, filled triangle (▴).

FIGS. 3A-3B show photomicrographs for J774A.1 cells cultured in 5% CO₂ for 2 days or 1% CO₂ for 5 days. FIG. 3A: 200X bright field of J774A.1 cells cultured in 5% CO₂ for 2 days. FIG. 3B: 200X phase-contrast of J774A.1 cells cultured in 1% CO₂ for 5 days. Arrows indicate representative morphology change of J774A.1 cells cultured in 1% CO₂.

FIGS. 4A-4B show ADCP assay assessed by flow cytometry with J774A.1 cells cultured in 5% CO₂ for 2 days, or 1% CO₂, for 3 days or 7 days. FIG. 4A: Representative flow cytometry quadrats data. FIG. 4B: Dose response curves for Daratumumab (0 to 1000 ng/ml, in duplicate): 5% CO₂ for 2 days, filled circle (●); 1% CO₂ for 3 days, filled triangle (▴); 1% CO₂ for 7 days, filled square (▪)

FIG. 5 shows ADCP assay in 96-well plate format with J774A.1 cells cultured in 5% CO₂ for 3 days, or 1% CO₂, for 7 days, 11 days, or 15 days. Dose response for Daratumumab (0 to 5000 ng/mL, in duplicate): 5% CO₂ for 3 days, filled circle (●); 1% CO₂ for 7 days, filled triangle (▴); 1% CO₂ for 11 days, filled square (▪); 1% CO₂ for 15 days, open circle (○).

FIG. 6 shows ADCP assay in 96-well plate format with J774A.1 cells cultured in 1% CO₂ for 5 days. Dose response for Daratumumab or Rituximab (0 to 1000 ng/ml): Daratumumab, filled circle (●); Rituximab, filled triangle (▴).

FIG. 7 shows ADCP assay in 96-well plate format with J774A.1 cells cultured in 1% CO₂ for 5 days. Dose response for Daratumumab (0 to 1000 ng/mL) that was freshly thawed or stored at 37 °C for 3 months or 6 months: freshly thawed, filled circle (●); 37 °C for 3 months, filled triangle (▴); 37 °C for 6 months, filled square (▪).

FIGS. 8A, 8B and 8C show that a different gene expression is observed between J774A.1 cells cultured in 1% CO₂ vs J774A.1 cells grown in 5% CO₂. FIG. 8A: Representative PCA between Group A (1% CO₂) and Group B (5% CO₂). FIG. 8B: IPA Graphical Summary illustrated upregulated immune system pathways in J774A.1 cells in 1% CO₂. In addition, the corresponding upstream regulators such as cytokine and transcription factors were also activated. In the figure, HMOX1, TRAF3 and HR were downregulated genes. The rest of the genes shown in the Figure were upregulated. FIG. 8C: Clustering of gene expression related to macrophage cell morphology, characteristics, and phagocytosis.

### DETAILED DESCRIPTION

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a cell” means one cell or more than one cell.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

As used herein, “antibody-dependent cellular phagocytosis” (ADCP) refers to a mechanism of elimination of antibody-bound target cells by internalization by phagocytic cells, e.g., macrophages generated in vitro from peripheral blood mononuclear cell (PBMC) preparations derived from human donors or other species (e.g., monkey and mouse, etc.) and also macrophage cell lines, e.g., J774A.1 cells (ATCC® TIB67™) and other macrophage-like phagocytic cell lines known by those skilled in the art. In a particular preferred embodiment, the phagocytic cells are J774A.1 cells.

The phagocytosis “signal” can be measured, for example, by using microscopy imaging techniques, flow cytometry, or a plate reader for tracking target cells, e.g., target cells labeled with fluorescent tags or dyes known by those skilled in the art. In a particular preferred embodiment, the target cells are labeled with the fluorescent dye pHrodo-Red (Invitrogen) and the phagocytosis signal is measured by flow cytometry (Aziz, Yang, & Wang, 2013) or with a plate reader, e.g., SpectraMax® Paradigm® Multi-Mode Microplate Reader (Molecular Devices). The phagocytosis signal can also be corrected by subtracting background, e.g., control with no antibody added in the ADCP assay.

As used herein, “target cells” can be tumor cells derived from cancer patients (e.g., B cell leukaemia cells, lymphoma cells, multiple myeloma cells), or tumor cell lines such as, e.g., Daudi cells, Ramos cells, Raji cells, and other tumor cell lines known by those skilled in the art. In a particular preferred embodiment, the target cells are Daudi cells (ATCC® CCL-213™).

The phrase “percent (%) CO₂” as used herein means percent (v/v) CO₂.

The phrase “percent (%) CO₂ atmosphere” as used herein means an atmosphere having a stated percent (v/v) CO₂.

The terms “antibody” and “antibodies” as used herein are meant in a broad sense and include immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

The term “antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a domain antibody (dAb) fragment, which consists of a VH domain. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

The phrase “isolated antibody” refers to an antibody or antibody fragment that is substantially free of other antibodies having different antigenic specificities (e.g., an isolated antibody specifically binding CD38 is substantially free of antibodies that specifically bind antigens other than human CD38). An isolated antibody that specifically binds CD38, however, can have cross-reactivity to other antigens, such as orthologs of human CD38, such as Macaca fascicularis (cynomolgus monkey) CD38. Moreover, an isolated antibody may be substantially free of other cellular material and/or chemicals.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. A human antibody may also contain amino acid differences when compared to the human germline or rearranged immunoglobulin sequences due to for example naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange such as bispecific antibodies.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” as used herein refers to a polypeptide or a polynucleotide that differs from a reference polypeptide or a reference polynucleotide by one or more modifications for example, substitutions, insertions or deletions.

“Synergy”, “synergism” or “synergistic” mean more than the expected additive effect of a combination.

The term “in combination with” as used herein means that two or more therapeutics can be used together in a mixture, concurrently as single agents or sequentially as single agents in any order.

The terms “treat” or “treatment” refers to both therapeutic treatment and prophylactic or preventative measures, wherein the object is to prevent or slow down (lessen) an undesired physiological change or disorder, such as the development or spread of tumor or tumor cells. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Inhibits growth” (e.g. referring to cells, such as tumor cells) refers to a measurable decrease in the cell growth in vitro or in vivo when contacted with a therapeutic or a combination of therapeutics or drugs when compared to the growth of the same cells grown in appropriate control conditions well known to the skilled in the art. Inhibition of growth of a cell in vitro or in vivo may be at least about 10%, 20%, 30%, 40%, 50%, 60%, 70%, 80%, 90%, 99%, or 100%. Inhibition of cell growth can occur by a variety of mechanisms, for example by antibody-dependent cell-mediated cytotoxicity (ADCC), antibody-dependent cellular phagocytosis (ADCP), complement dependent cytotoxicity (CDC), apoptosis, necrosis, or by inhibition of cell proliferation.

A “therapeutically effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction of a tumor burden, arrested or slowed growth of a tumor, and/or absence of metastasis of cancer cells to other locations in the body.

An exemplary antibody used in the ADCP assays in the examples, Daratumumab (HUMAX®-CD38), is a human monoclonal antibody that binds human CD38, see, e.g., U.S. Pat. No. 7,829,673 and (de Weers et al., 2011). Daratumumab has been shown to have positive effects against multiple myeloma. Another exemplary antibody used in the ADCP assays in the examples, Rituximab (Rituxan®), is a chimeric anti-CD20 antibody targeting the CD20 protein which is expressed on over 95% of B cell lymphomas. Monoclonal antibody therapy with Rituximab has been shown to be an effective treatment of B-cell lymphomas, such as non-Hodgkin's lymphoma, see, e.g., U.S. Pat. No. 8,557,244.

In some embodiments of any of the compositions or methods described herein, a range is intended to comprise every integer or fraction or value within the range.

Embodiments described herein as “comprising” one or more features may also be considered as disclosure of the corresponding embodiments “consisting of” and/or “consisting essentially of” such features.

### Method for Increasing ADCP Activity

Provided is a method for increasing antibody-dependent cellular phagocytosis (ADCP) activity of a phagocytic cell in a sample comprising: culturing the phagocytic cell in a low percent (%) CO₂ atmosphere for a period of time sufficient to increase ADCP activity of the phagocytic cell.

In some embodiments, the low % CO₂ is from about 0.1 % CO₂ to about 1% CO₂. In further embodiments, the low % CO₂ is about 1% CO₂. In some embodiments, the low % CO₂ is 0.1, 0.2, 0.3, 0.4, 0.5, 0.6, 0.7, 0.8, 0.9, or 1% CO₂.

In some embodiments, the low % CO₂ atmosphere is maintained by CO₂ being infused into a humidified chamber or incubator.

In some embodiments, the period of time sufficient to increase ADCP activity of the phagocytic cells is 3 to 15 days, for example 3, 4, 5, 6, 7, 8, 9, 10, 11, 12, 13, 14, or 15 days. In further embodiments, the period of time sufficient to increase ADCP activity of the phagocytic cell is 3 to 7 days. In yet further embodiments, the period of time sufficient to increase ADCP activity of the phagocytic cell is 3, 4, 5, 6, or 7 days.

In some embodiments, the method further comprises determining the ADCP activity of the phagocytic cell using target cells labeled with a fluorescent dye.

In some embodiments, the phagocytic cell is a J774A.1 cell or a donor cell. J774A.1 cells (ATCC® TIB67™) are a mostly adherent monocyte/macrophage cell line derived from mice (Ralph, Moore, & Nilsson, 1976) that are active in antibody dependent phagocytosis ((Ralph & Nakoinz, 1975). Donor cells may be obtained from a human subject. In further embodiments, the donor cell is a human macrophage cell. In some embodiments, the human macrophage cell is derived from an effusion fluid, for example a peritoneal exudate (U.S. Pat. No. 8,975,040).

In some embodiments, the phagocytic cells are cultured in the presence of target cells. In further embodiments, target cells are added to the phagocytic cell culture after about 1 to about 14 days. In yet further embodiments, target cells are added to the phagocytic cell culture after about 1, 2, 3, 4, 5, 6, 7, 8, 9, 10, 11, 12, 13, or 14 days. In certain embodiments, target cells are added to the phagocytic cell culture after about 7 days. In further embodiments, the target cells are Daudi cells, B cells, leukemia cells or lymphoma cells. In yet further embodiments, the target cells are Daudi cells. Daudi cells (ATCC® CCL213™) are a well characterized B lymphoblast cell line derived from a 16-year-old black male with Burkitt's lymphoma.

In some embodiments, the fluorescent dye is pHrodo-Red, pHAb or AcidiFluor.

In some embodiments, expression of a regulator of ADCP is increased. In further embodiments, the regulator is ATF4, FOXO3, IL1B, IL6, VEGFA, HGF, EGF, CHD1, SELP, TIMP3, DACH1, STAT3, GLI1, SP3, or combinations thereof.

In some embodiments, expression of a regulator of ADCP is decreased. In further embodiments, the regulator is TP53, TNF, TGFB1, STAT6, MYD88, HRAS or combinations thereof.

In some embodiments, the target cells are contacted with an antibody or fragment thereof.

In some embodiments, the sample is cultured in a humidified chamber or incubator.

### Assaying ADCP Activity

Also provided is a method for assaying ADCP activity of a phagocytic cell in a sample comprising: culturing the phagocytic cell in a low percent (%) CO₂ atmosphere for a period of time sufficient to increase ADCP activity of the phagocytic cell; and detecting ADCP activity of the phagocytic cell in the sample.

In some embodiments, the low % CO₂ is from about 0.1 % CO₂ to about 1% CO₂. In some embodiments, the low % CO₂ is 0.1, 0.2, 0.3, 0.4, 0.5, 0.6, 0.7, 0.8, 0.9, or 1% CO₂.

In some embodiments, the low % CO₂ is 1% CO₂.

In some embodiments, the low % CO₂ atmosphere is maintained by CO₂ being infused into a humidified chamber or incubator.

In some embodiments, the period of time sufficient to increase ADCP activity of the phagocytic cells is 3 to 15 days, for example 3, 4, 5, 6, 7, 8, 9, 10, 11, 12, 13, 14, or 15 days. In further embodiments, the period of time sufficient to increase ADCP activity of the phagocytic cell is 3 to 7 days. In yet further embodiments, the period of time sufficient to increase ADCP activity of the phagocytic cell is 3, 4, 5, 6, or 7 days.

In some embodiments, the method further comprises determining the ADCP activity of the phacobytic cell using target cells labeled with a fluorescent dye.

In some embodiments, the phagocytic cell is a J774A.1 cell or a donor cell. J774A.1 cells (ATCC® TIB67™) are a mostly adherent monocyte/macrophage cell line derived from mice (Ralph, Moore, & Nilsson, 1976) that are active in antibody dependent phagocytosis (Ralph & Nakoinz, 1975). Donor cells may be obtained from a human subject. In further embodiments, the donor cell is a human macrophage cell.

In some embodiments, the phagocytic cells are cultured in the presence of target cells. In further embodiments, target cells are added to the phagocytic cell culture after about 3 to about 10 days, for example after about 7 days.

In some embodiments, the target cells are labeled with a fluorescent dye.

In some embodiments, the phagocytic cell is a J774A.1 cell or a donor cell. J774A.1 cells (ATCC® TIB67™) are a mostly adherent monocyte/macrophage cell line derived from mice (Ralph, Moore, & Nilsson, 1976) that are active in antibody dependent phagocytosis (Ralph & Nakoinz, 1975). Donor cells may be obtained from a human subject. In further embodiments, the donor cell is a human macrophage cell.

In some embodiments, the target cells are Daudi cells, B cells, leukemia cells or lymphoma cells. In yet further embodiments, the target cells are Daudi cells. Daudi cells (ATCC® CCL 213™) are a well characterized B lymphoblast cell line derived from a 16-year-old black male with Burkitt's lymphoma.

In some embodiments, the fluorescent dye is pHrodo-Red, pHAb or AcidiFluor.

In some embodiments, expression of a regulator of ADCP is increased. In further embodiments, the regulator is ATF4, FOXO3, IL1B, IL6, VEGFA, HGF, EGF, CHD1, SELP, TIMP3, DACH1, STAT3, GLI1, SP3, or combinations thereof.

In some embodiments, expression of a regulator of ADCP is decreased. In further embodiments, the regulator is TP53, TNF, TGFB1, STAT6, MYD88, HRAS or combinations thereof.

In some embodiments, the target cells are contacted with an antibody or fragment thereof.

In some embodiments, the sample is cultured in a humidified chamber or incubator.

### Assaying ADCP Activity

Provided is a method for assaying ADCP activity of a phagocytic cell in a sample comprising: culturing the phagocytic cell in a low percent (%) CO₂ atmosphere for a period of time sufficient to increase ADCP activity of the phagocytic cell; and detecting ADCP activity of the phagocytic cell in the sample.

In some embodiments, the low % CO₂ is from about 0.1% CO₂ to about 1% CO₂. In some embodiments, the low % CO₂ is 0.1, 0.2, 0.3, 0.4, 0.5, 0.6, 0.7, 0.8, 0.9, or 1% CO₂.

In some embodiments, the low % CO₂ is 1% CO_{2.}

In some embodiments, the low % CO₂ atmosphere is maintained by CO2 being infused into a humidified chamber or incubator.

In some embodiments, the period of time sufficient to increase ADCP activity of the phagocytic cells is 3 to 15 days, for example 3, 4, 5, 6, 7, 8, 9, 10, 11, 12, 13, 14, or 15 days. In further embodiments, the period of time sufficient to increase ADCP activity of the phagocytic cell is 3 to 7 days. In yet further embodiments, the period of time sufficient to increase ADCP activity of the phagocytic cell is 3, 4, 5, 6, or 7 days.

In some embodiments, the method further comprises determining the ADCP activity of the phacobytic cell using target cells labeled with a fluorescent dye.

In some embodiments, the phagocytic cell is a J774A.1 cell or a donor cell. J774A.1 cells (ATCC® TIB67™) are a mostly adherent monocyte/macrophage cell line derived from mice (Ralph, Moore, & Nilsson, 1976) that are active in antibody dependent phagocytosis (Ralph & Nakoinz, 1975). Donor cells may be obtained from a human subject. In further embodiments, the donor cell is a human macrophage cell.

In some embodiments, the phagocytic cells are cultured in the presence of target cells. In further embodiments, target cells are added to the phagocytic cell culture after about 3 to about 10 days, for example after about 7 days.

In some embodiments, the target cells are labeled with a fluorescent dye.

In some embodiments, the phagocytic cell is a J774A.1 cell or a donor cell. J774A.1 cells (ATCC® TIB67™) are a mostly adherent monocyte/macrophage cell line derived from mice (Ralph, Moore, & Nilsson, 1976) that are active in antibody dependent phagocytosis (Ralph & Nakoinz, 1975). Donor cells may be obtained from a human subject. In further embodiments, the donor cell is a human macrophage cell.

In some embodiments, the target cells are Daudi cells, B cells, leukemia cells or lymphoma cells. In yet further embodiments, the target cells are Daudi cells. Daudi cells (ATCC® CCL 213™) are a well characterized B lymphoblast cell line derived from a 16-year-old black male with Burkitt's lymphoma.

In some embodiments, the fluorescent dye is pHrodo-Red, pHAb or AcidiFluor.

In some embodiments, expression of a regulator of ADCP is increased. In further embodiments, the regulator is ATF4, FOXO3, IL1B, IL6, VEGFA, HGF, EGF, CHD1, SELP, TIMP3, DACH1, STAT3, GLI1, SP3, or combinations thereof.

In some embodiments, expression of a regulator of ADCP is decreased. In further embodiments, the regulator is TP53, TNF, TGFB1, STAT6, MYD88, HRAS or combinations thereof.

In some embodiments, the target cells are contacted with an antibody or fragment thereof.

In some embodiments, the sample is cultured in a humidified chamber or incubator.

### EXAMPLES

### Materials and Methods

### Human Donor Macrophages

Human donor macrophages were prepared by standard methods as known by those skilled in the art. In brief, frozen PBMC were enriched by Human monocyte enrichment cocktail without CD 16 depletion (STEMCELL Technology® 19058). Monocytes were cultured in X-VIVO10 Medium (Lonza® 04-380Q) supplemented with 10% fetal bovine serum (FBS) and 25 ng/ml M-CSF at 37 °C and 5% CO₂. At day 3-4, 50% medium were replaced with fresh X-VIVO10 +10% FBS. At day 6, IFN-γ (50 ng/mL) were added and cells were harvested for ADCP assay at day 7.

### J774A.1 Cells

J774A.1 cells (ATCC® TIB 67™) are a mostly adherent monocyte/macrophage cell line derived from mice (Ralph, Moore, & Nilsson, 1976) that are active in antibody dependent phagocytosis (Ralph & Nakoinz, 1975). J774A.1 cells were cultured in cultured in Dulbecco's Modified Eagle's Medium (DMEM), supplemented with FBS to a final concentration of 10%.

Subcultures are prepared by cell dissociation reagent, Accutase (eBioscience, 00-4555-56) and scraping. For parental J774A.1, cells are cultured in 5% CO₂ at 37 °C. For low CO₂ is noted in the Figures and Examples, J774A.1 cells were seeded at 8×10⁶ cell/40 mL in a T150 flask and incubated in low CO₂ for the period of time indicated.

### Daudi Cells

Daudi cells (ATCC® CCL213™) are a well characterized B lymphoblast cell line derived from a 16-year-old black male with Burkitt's lymphoma. The Daudi cells were cultured in suspension in RPMI-1640 medium (ATCC 30-2001), with FBS added to a final concentration of 10%, at 37 °C and 5% CO₂. Fresh medium was added every 2 to 3 days (depending on cell density) until the day of the ADCP assay.

### Labeling Cells

Target cells (e.g., Daudi cells) were washed twice with phosphate buffered saline (PBS) and resuspended in PBS at 10⁶ cells/ml. 25 μl of 1 mg/ml pHrodo-Red (stock solution in DMSO) was added per 50 ml of cell suspension (i.e., final concentration 500 ng/ml and 10⁶ cells pHrodo-Red). After incubation for 30 min at 37 °C, cells were washed twice with PBS and resuspended in DMEM medium at 5×10⁶ cells/ml before use in ADCP assay. pHrodo-Red SE was from Invitrogen (Waltham, MA; P36600).

Macrophages (e.g., human donor macrophages and J774A.1 cells) were stained with a rat anti-Mouse CD11b antibody [M1/70] labelled with Alexa Fluor® 488 (Biolegend, San Diego, CA). The macrophages were lifted from a culture flask with Accutase for 30 min at 37 °C and washed two times with PBS and 2.5 μl/mlof the anti-Mouse CD11b antibody were added for 20 min at 4 °C. This staining provided a homogenous surface staining of macrophages and was also useful to distinguish cell surfaces of cells during flow cytometry.

### Flow Cytometry

For flow cytometry, data were acquired on an FACScan Flow Cytometer (BD Biosciences, San Jose, CA) and analyzed with FlowJo software (Flowjo, LLC, Ashland, OR).

### 96-Well Plate Format Read by Flow Cytometry and Microplate Reader

For ADCP assay done in 96-well plates (Corning, 3799) for flow cytometry, serial diluted Daratumumab or Rituximab were pre-incubated with 2.5×10⁴ of pHrodo-red labeled Daudi cells in each well for 15 minutes at room temperature. Then, 1×10⁵ human macrophages or J774A.1 cells were added and mixed. The plate were centrifuge at 40 xg for 1 minute with minimal acceleration and deceleration and incubated in 37 °C incubator with 5% or 1% CO₂ for 3 or 24 hours. The cells then wash once with DPBS and detached with Accutase for further CD11b staining and flow cytometry. For microplate reader, serial diluted Daratumumab or rituximab were pre-incubated with 1.25×10⁴ of pHrodo-red labeled Daudi cells in each well (Corning, 3904) for 15 minutes at room temperature. Then, 5×10⁴ human macrophages or J774A.1 cells were added and mixed. The plate were centrifuge at 40 xg for 1 minute with minimal acceleration and deceleration and incubated in 37 °C incubator with 1% CO₂ for 2.5 hours. Data was acquired using a SpectraMax® Paradigm® Multi-Mode Microplate Reader (Molecular Devices, San Jose, CA) at Ex/Em 560/600 nm with well scan mode. Results were plotted after the subtraction of the signal from wells with no antibody added (subtracting signal from background/control).

### Example 1: ADCP by Flow Cytometry With Human Donor Macrophages

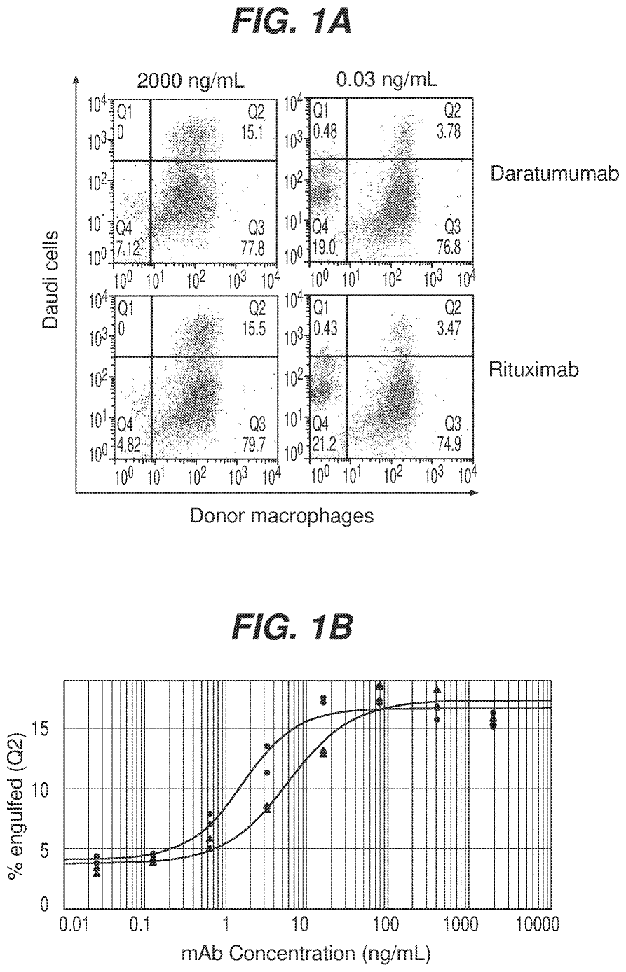

Human donor macrophages (1×10⁵) were incubated with pHrodo-red labeled Daudi cells (2.5×10⁴) and titrated monoclonal antibodies (Daratumumab or Rituximab, 0.03 to 2000 ng/ml) for 3 hours in a 96-well plate. Cells were detached from the wells and stained with an anti-CD 11b-A488 antibody. ADCP was assessed by flow cytometry and analyzed by FlowJo software and dose response curves were generated by plotting percentage for cells of Q2 gating for pHrodo-red Daudi cells engulfed by human donor macrophages (FIGS. 1A-1B).

The results verified that daratumumab and rituximab can induce ADCP of tumor cells in human macrophages. ADCP flow cytometry analysis showed that Daudi cells were opsonized by human macrophages in the present of either daratumumab or rituximab. In addition, Daratumumab and rituximab dose dependent response curves were observed in human macrophage that engulfed the pHrodo labeled Daudi cells.

### Example 2: ADCP by Flow Cytometry With J774A.1 Cells Cultured in 5% Co₂

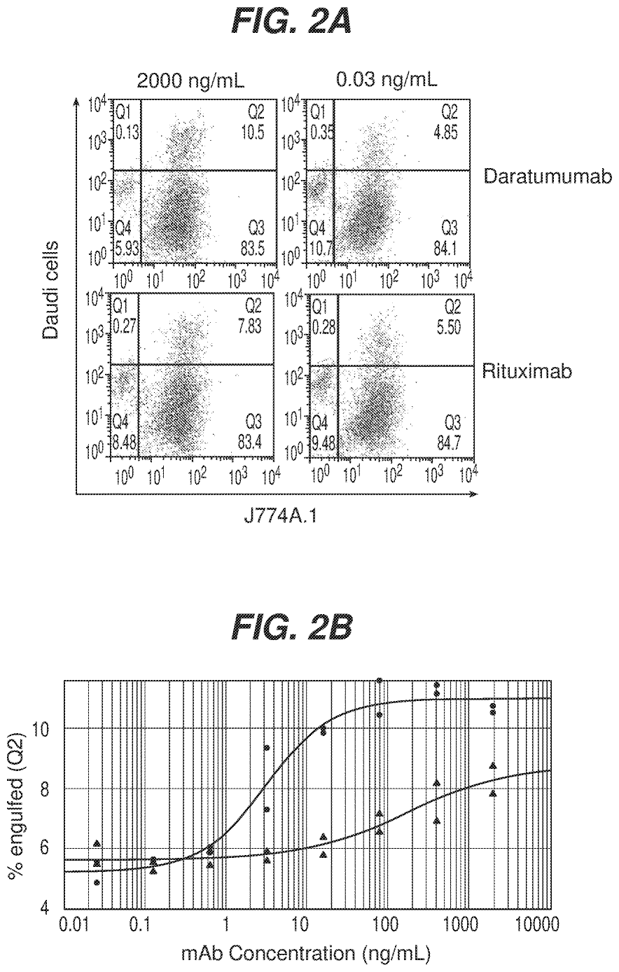

J774A.1 cells (1×10⁵) that had been maintained in 5% CO₂, were incubated with pHrodo-red labeled Daudi cells (2.5×10⁴) and titrated monoclonal antibodies (Daratumumab or Rituximab, 0.03 to 2000 ng/ml) for 24 hours in a 96-well plate. Cells were detached from the wells and stained with an anti-CD11b-A488 antibody. ADCP was assessed by flow cytometry analyzed by FlowJo software (FlowJo, LLC, Ashland, OR) and dose response curves were generated by plotting percentage for cells of Q2 gating for pHrodo-red Daudi cells engulfed by J774A.1 cells (FIGS. 2A-2B).

The results supported that daratumumab and rituximab can induce ADCP of tumor cells in mouse macrophage cell line, J774A.1. ADCP flow cytometry analysis showed that Daudi cells were opsonized by J744A.1 in the presence of either daratumumab or rituximab. The % of engulfment from J774A.1 cell line was less than human macrophages, which suggested J774A.1 cell line has lower ADCP activity. A less dynamic daratumumab and rituximab dose dependent response curves were observed in J774A.1 cells that engulfed the pHrodo labeled Daudi cells.

### Example 3: Morphology Change for J774A.1 Cells Cultured in 1% Co₂

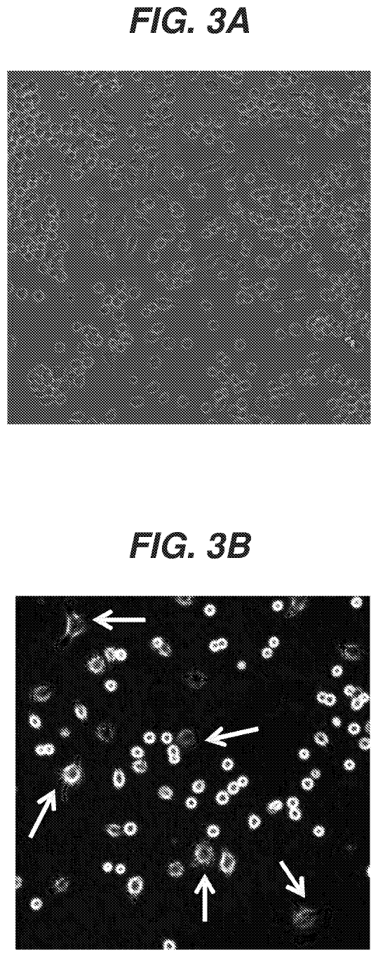

J774A.1 cells were sub-cultured in 5% CO₂ humidified chamber for 2 days or in 1% CO₂ humidified chamber for 5 days. Photos were taken by microscopy, 200× bright field or 200× phase-contrast in (FIGS. 3A-3B) .

Bright field microcopy showed morphological change after J774A.1 cells were cultured in 1% CO₂ for 5 days. Furthermore, the spreading of J744A.1 cells with macrophage morphology were more than round shape J744A.1 cells, when they were cultured in 1% CO₂.

### Example 4: Increased ADCP for J774A.1 Cells Cultured in 1% Co₂

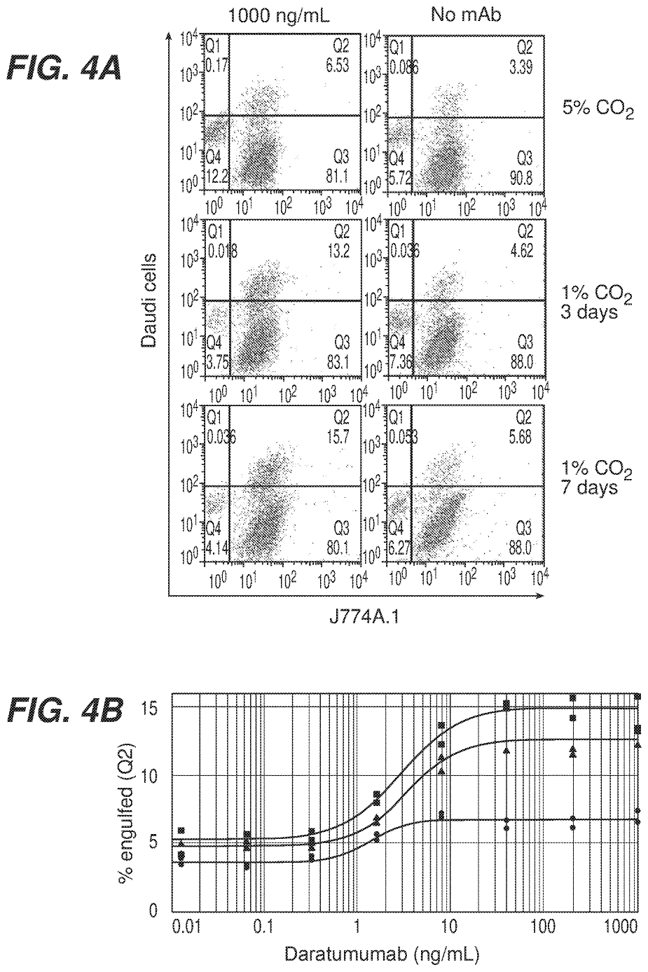

J 774A.1 cells were cultured in a 5% CO₂ humidified chamber for 2 days or cultured in a 1% CO₂ humidified chamber for 3 days or 7 days. The J774A.1 cells (1×10⁵) were incubated with pHrodo-red labeled Daudi cells (2.5×10⁴) and titrated monoclonal antibody (Daratumumab, 0 to 1000 ng/mL) for 24 hours in a 96-well plate. Cells were detached from the wells and stained with an anti-CD11b-A488 antibody. ADCP was assessed by flow cytometry analyzed by FlowJo software and dose response curves were generated by plotting percentage for cells of Q2 gating for pHrodo-red Daudi cells engulfed by J774A.1 cells (FIGS. 4A-4B).

The data demonstrated that 1% CO₂ cell culture condition enhances ADCP activity in J744A.1 cells. ADCP results were compared between J744A.1 cells that were cultured in 5% CO₂ for 2 days, 1% CO₂ for 3 days, and 1% CO₂ for 7 days, respectively. ADCP flow cytometry analysis showed that Dauid cells, in the presence of daratumumab, were opsonized by J744A.1 in a higher level in 1% CO₂ than 5% CO₂ culturing conditions. The % engulfment from J774A.1 cells that were cultured in 1% CO₂ was higher than J774A.1 cells that were cultured in 5% CO₂. A good dynamic daratumumab dose dependent response curves were observed with J774A.1 cells that were cultured in 1% CO₂.

### Example 5: Effect of Time on ADCP for J774A.1 Cells Cultured in 1% Co₂

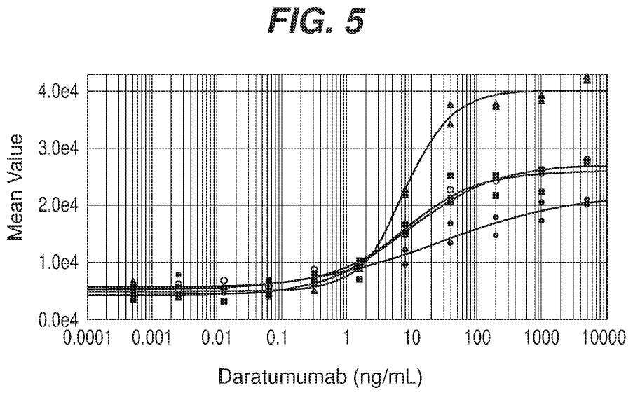

J774A.1 cells were cultured in 5% CO2 humidified chamber for 3 days or 1% CO₂ humidified chamber for 7 days, 11 days, or 15 days. J774A.1 cells (5×10⁴) were then incubated with pHrodo-red labeled Daudi cells (1.25×10⁴) for 2 hours in a 96-well plate with titrated monoclonal antibody (Daratumumab, 0 to 5000 ng/ml). ADCP was assessed by microplate reader and the results were plotted after subtraction of the no antibody control (FIG. 5).

The results showed that longer culture time of J774A.1 cells in 1% CO₂ may not enhance ADCP activity. Daratumumab dose dependent response curves that had the lowest fluorescent signal to noise ratio was with J744A.1 cells in 5% CO₂. And daratumumab dose dependent response curves that had the highest fluorescent signal to noise ratio was with J744A.1 cells in 1% CO₂ for 7 days. Prolonged (i.e. day 11 and day 15) 1% CO₂ cultured condition did not increase fluorescent signal to noise ratio nor increase the curve dynamic.

### Example 6: Dose Dependent ADCP With Daratumumab or Rituximab

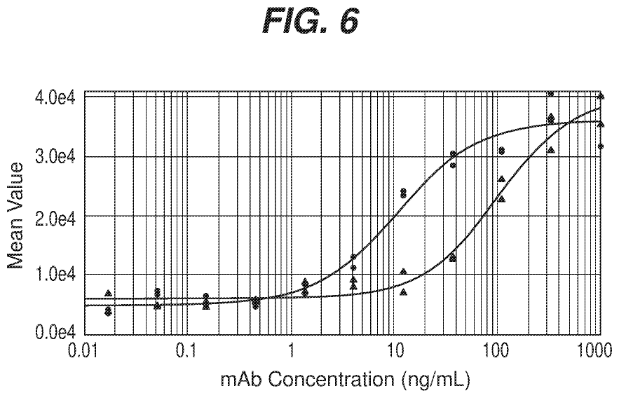

J774A.1 cells were cultured in 1% CO₂ humidified chamber for 5 days. J774A.1 cells (5×10⁴) were then incubated with pHrodo-red labeled Daudi cells (1.25×10⁴) for 2.5 hours in a 96-well plate with titrated antibodies (Daratumumab or Rituximab, 0 to 1000 ng/ml). ADCP was assessed by microplate reader and the results were plotted after subtraction of the no antibody control (FIG. 6).

The results demonstrated that both daratumumab and rituximab can induce ADCP of tumor cells in mouse macrophage cell line, J774A.1 that was cultured in 1% CO₂. Daratumumab and rituximab dose dependent response curves were observed in J774A.1 that engulfed the pHrodo labeled Daudi cells, which showed ADCP activity.

### Example 7: Stability of Daratumumab by ADCP

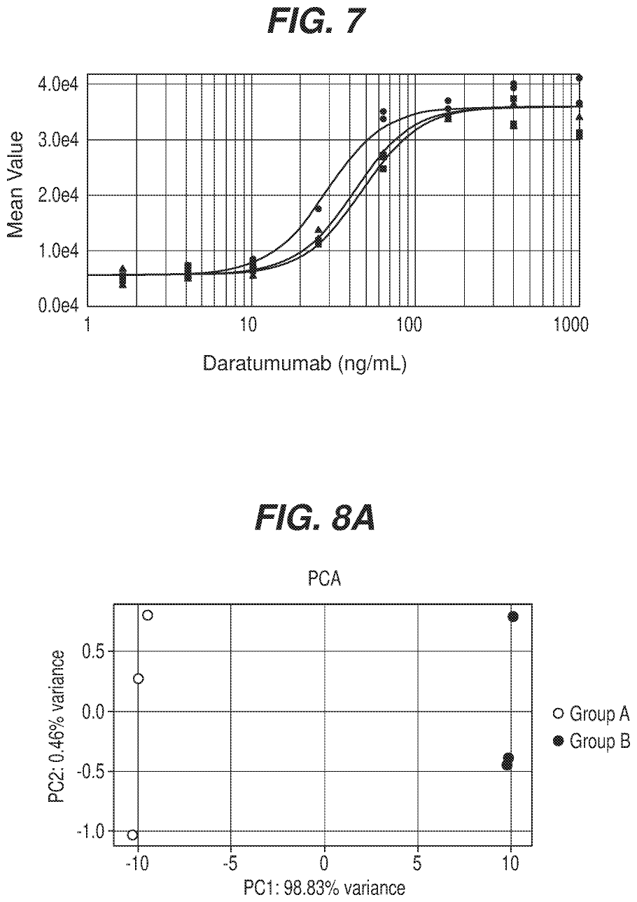

J774A.1 cells were cultured in 1% CO₂ humidified chamber for 5 days. J774A.1 cells (5×10⁴) were then incubated with pHrodo-red labeled Daudi cells (1.25×10⁴) for 2.5 hours in a 96-well plate with titrated Daratumumab (0 to 1000 ng/mL) that was freshly thawed or stored at 37 °C for 3 months or 6 months. ADCP as assessed by microplate reader and the results were plotted after subtraction of the no antibody control (FIG. 7).

The data demonstrated stability indicating property of the ADCP assay with J744A.1 cells cultured in 1% CO₂. The dose response curves from daratumumab stability samples, that were stored at 37 °C for 3 and 6 months, were shifted right as compared to the freshly thawed sample. The results showed that these thermal exposed samples had lower potencies (i.e. <100%).

### Example 8: RNA-Seq Analysis

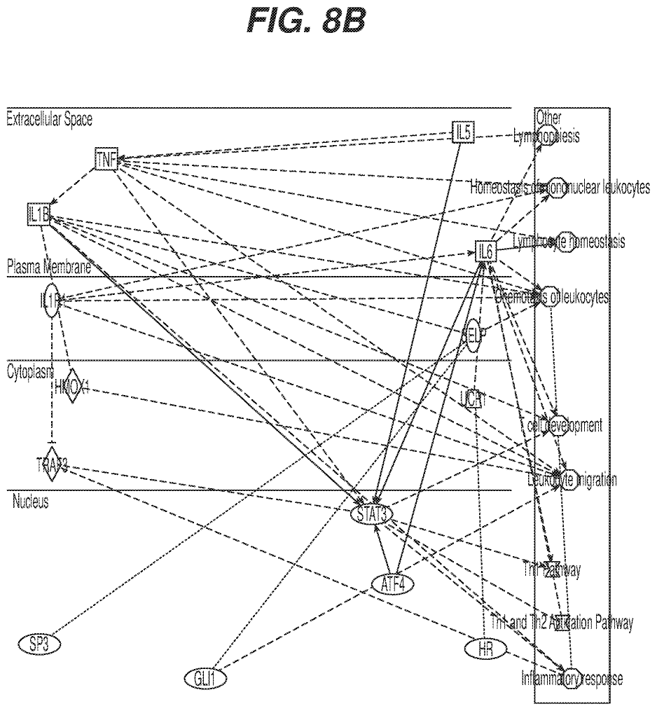

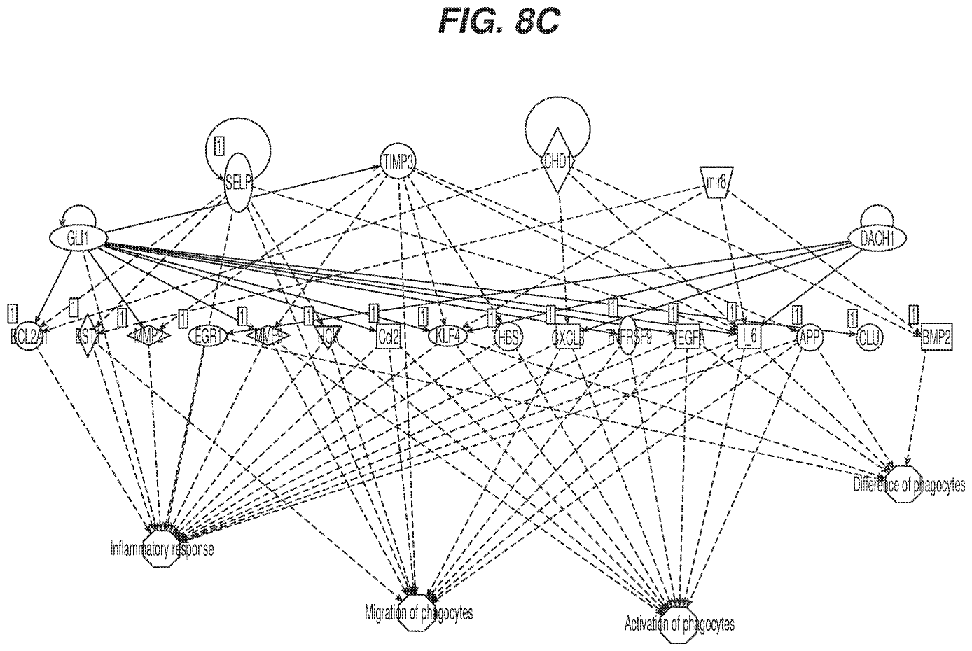

Two groups of J774A.1 cells were incubated with different CO₂ concentrations (1% vs. 5%). J774A.1 cells, 1×10⁶ and 4×10⁶ cells/mL were cultured in either 5% or 1% CO₂ humidified chamber for 4 days, respectively. RNA from the corresponding culture conditions was extracted for RNA-Seq Analysis (FIGS. 8A, 8B, and 8C). PCA (Principal components analysis) demonstrated that gene expression profiles between J744A.1 cells cultured in 1% CO₂ vs 5% CO₂ were significantly different. IPA (Ingenuity Pathways Analysis) Summary showed that several key upstream regulators of the immune system pathway such as several cytokines (i.e TNF, IL6), and transcription factors (i.e. ATF4, STAT3) were upregulated in J744A1 cells in 1% CO₂. The activation of these upstream regulators may contribute to J744A.1 cells acquiring a more macrophage-like morphology that exhibit enhanced phagocytosis activity.

### Conclusion

As described supra, ADCP activity surprisingly and unpredictably increased for J774A.1 cells cultured in 1% CO₂. The increased ADCP activity provided an improved ADCP assay with increased signal compared to background. The improved ADCP assay method is suitable for determining ADCP activity of an antibody, for comparing the ADCP activity of different antibodies or different antibody preparations, and for measuring the stability of an antibody as is required for quality control during production or storage of an antibody.

### Embodiments

The following exemplary embodiments further describe optional aspects of the presently disclosed technology and are part of the Detailed Description. These examplary embodiments are set forth in a format substantially akin to claims (each with numerical designations followed by a capital letter), although they are not technically claims of the present application. The following exemplary embodiments refer to each other in dependent relationships as “embodiments” instead of “claims.”
1A. A method for increasing antibody-dependent cellular phagocytosis (ADCP) activity of a phagocytic cell in a sample comprising: culturing the phagocytic cell in a low percent (%) CO2 atmosphere for a period of time sufficient to increase ADCP activity of the phagocytic cell.
2A. The method of embodiment 1A, wherein the low % CO₂ is from about 0.1 % CO₂ to about 1% CO₂.
3A. The method of embodiment 2A, wherein the low % CO₂ is about 1% CO₂.
4A. The method of any one of embodiments 1A-3A, wherein the period of time sufficient to increase ADCP activity of the phagocytic cells is 3 to 15 days.
5A. The method of any one of embodiments 1A-4A, wherein the period of time sufficient to increase ADCP activity of the phagocytic cells is 3 to 7 days
6A. The method of any one of embodiments 1A-5A, further comprising determining the ADCP activity of the phagocytic cell using target cells labeled with a fluorescent dye.
7A. The method of any one of embodiments 1A-6A, wherein the phagocytic cell is a J774A.1 cell or a donor cell.
8A. The method of any one of embodiments 1A-7A, wherein the phagocytic cells are cultured in the presence of target cells.
9A. The method of embodiment 8A, wherein target cells are added to the phagocytic cell culture after about 7 days.
10A. The method of any one of embodiments 8A-9A, wherein the target cells are Daudi cells, B cells, leukemia cells or lymphoma cells.
11A. The method of any one of embodiments 6A-10A, wherein the fluorescent dye is pHrodo-Red, pHAb or AcidiFluor.
12A. The method of any one of embodiments 1A-11A, wherein expression of a regulator of ADCP is increased.
13A. The method of embodiment 12A, wherein the regulator is ATF4, FOXO3, IL1B, IL6, VEGFA, HGF, EGF, CHD1, SELP, TIMP3, DACH1, STAT3, GLI1, SP3, or combinations thereof.
14A. The method of any one of embodiments 1A-11A, wherein expression of a regulator of ADCP is decreased.
15A. The method of embodiment 14A, wherein the regulator is TP53, TNF, TGFB1, STAT6, MYD88, HRAS or combinations thereof.
16A. The method of any one of embodiments 8A-15A, wherein the target cells are contacted with an antibody or fragment thereof.
17A. The method of any one of embodiments 1A-16A, wherein the sample is cultured in a humidified chamber or incubator.
18A. A method for assaying ADCP activity of a phagocytic cell in a sample comprising: culturing the phagocytic cell in a low percent (%) CO₂ atmosphere for a period of time sufficient to increase ADCP activity of the phagocytic cell; and detecting ADCP activity of the phagocytic cell in the sample.
19A. The method of embodiment 18A, wherein the low % CO₂ is from about 0.1 % CO₂ to about 1% CO₂.
20A. The method of embodiment 19A, wherein the low % CO₂ is about 1% CO₂.
21A. The method of any one of embodiments 18A-20A, wherein the period of time sufficient to increase ADCP activity of the phagocytic cells is 3 to 15 days.
22A. The method of any one of embodiments 18A-21A, wherein the period of time sufficient to increase ADCP activity of the phagocytic cells is 3 to 7 days
23A. The method of any one of embodiments 18A-22A, further comprising determining the ADCP activity of the phacobytic cell using target cells labeled with a fluorescent dye.
24A. The method of any one of embodiments 18A-23A, wherein the phagocytic cell is a J774A.1 cell or a donor cell.
25A. The method of any one of embodiments 18A-24A, wherein the phagocytic cells are cultured in the presence of target cells.
26A. The method of embodiment 25A, wherein target cells are added to the phagocytic cell culture after about 7 days.
27A. The method of any one of embodiments 25A-26A, wherein the target cells are labeled with a fluorescent dye.
28A. The method of any one of embodiments 18A-27A, wherein the phagocytic cell is a J774A.1 cell or a donor cell.
29A. The method of any one of embodiments 25A-28A, wherein the target cells are Daudi cells, B cells, leukemia cells or lymphoma cells.
30A. The method of any one of embodiments 23A-29A, wherein the fluorescent dye is pHrodo-Red, pHAb or AcidiFluor.
31A. The method of any one of embodiments 18A-30A, wherein expression of a regulator of ADCP is increased.
32A. The method of embodiment 31A, wherein the regulator is ATF4, FOXO3, IL1B, IL6, VEGFA, HGF, EGF, CHD1, SELP, TIMP3, DACH1, STAT3, GLI1, SP3, or combinations thereof.
33A. The method of any one of embodiments 18A-32A, wherein expression of a regulator of ADCP is decreased.
34A. The method of embodiment 33A, wherein the regulator is TP53, TNF, TGFB1, STAT6, MYD88, HRAS or combinations thereof.
35A. The method of any one of embodiments 25A-34A, wherein the target cells are contacted with an antibody or fragment thereof.
36A. The method of any one of embodiments 18A-35A, wherein the sample is cultured in a humidified chamber or incubator.

### REFERENCES

Aziz, M., Yang, W. L., & Wang, P. (2013). Measurement of phagocytic engulfment of apoptotic cells by macrophages using pHrodo succinimidyl ester. Curr Protoc Immunol, Chapter 14, Unit 14 31. doi: 10.1002/0471142735.im1431s100
de Weers, M., Tai, Y. T., van der Veer, M. S., Bakker, J. M., Vink, T., Jacobs, D. C., . . . Parren, P. W. (2011). Daratumumab, a novel therapeutic human CD38 monoclonal antibody, induces killing of multiple myeloma and other hematological tumors. J Immunol, 186(3), 1840-1848. doi: 10.4049/jimmunol.1003032
Geissmann, F., Manz, M. G., Jung, S., Sieweke, M. H., Merad, M., & Ley, K. (2010). Development of monocytes, macrophages, and dendritic cells. Science, 327(5966), 656-661. doi: 10.1126/science.1178331
Herter, S., Birk, M. C., Klein, C., Gerdes, C., Umana, P., & Bacac, M. (2014). Glycoengineering of therapeutic antibodies enhances monocyte/macrophage-mediated phagocytosis and cytotoxicity. J Immunol, 192(5), 2252-2260. doi: 10.4049/jimmunol.1301249
Khagi, Y., & Mark, T. M. (2014). Potential role of Daratumumab in the treatment of multiple myeloma.Onco Targets Ther, 7, 1095-1100. doi: 10.2147/OTT.S49480
Mossner, E., Brunker, P., Moser, S., Puntener, U., Schmidt, C., Herter, S., . . . Umana, P. (2010). Increasing the efficacy of CD20 antibody therapy through the engineering of a new type II anti-CD20 antibody with enhanced direct and immune effector cell-mediated B-cell cytotoxicity. Blood, 115(22), 4393-4402. doi: 10.1182/blood-2009-06-225979
Oflazoglu, E., & Audoly, L. P. (2010). Evolution of anti-CD20 monoclonal antibody therapeutics in oncology. MAbs, 2(1), 14-19.
Overdijk, M. B., Verploegen, S., Bogels, M., van Egmond, M., Lammerts van Bueren, J. J., Mutis, T., . . . Parren, P. W. (2015). Antibody-mediated phagocytosis contributes to the anti-tumor activity of the therapeutic antibody Daratumumab in lymphoma and multiple myeloma. MAbs, 7(2), 311-321. doi: 10.1080/19420862.2015.1007813
Rafiq, S., Butchar, J. P., Cheney, C., Mo, X., Trotta, R., Caligiuri, M., . . . Byrd, J. C. (2013). Comparative assessment of clinically utilized CD20-directed antibodies in chronic lymphocytic leukemia cells reveals divergent NK cell, monocyte, and macrophage properties. J Immunol, 190(6), 2702-2711. doi: 10.4049/jimmunol.1202588
Ralph, P., Moore, M. A., & Nilsson, K. (1976). Lysozyme synthesis by established human and murine histiocytic lymphoma cell lines. J Exp Med, 143(6), 1528-1533.
Ralph, P., & Nakoinz, I. (1975). Phagocytosis and cytolysis by a macrophage tumour and its cloned cell line. Nature, 257(5525), 393-394.
Richards, J. O., Karki, S., Lazar, G. A., Chen, H., Dang, W., & Desjarlais, J. R. (2008). Optimization of antibody binding to FcgammaRIIa enhances macrophage phagocytosis of tumor cells. Mol Cancer Ther, 7(8), 2517-2527. doi: 10.1158/1535-7163.MCT-08-0201
Shuptrine, C. W., Surana, R., & Weiner, L. M. (2012). Monoclonal antibodies for the treatment of cancer. Semin Cancer Biol, 22(1), 3-13. doi: 10.1016/j. semcancer.2011.12.009
Ward, E. S., Gussow, D., Griffiths, A. D., Jones, P. T., & Winter, G. (1989). Binding activities of a repertoire of single immunoglobulin variable domains secreted from Escherichia coli. Nature, 341(6242), 544-546. doi: 10.1038/341544a0
Watanabe, M., Wallace, P. K., Keler, T., Deo, Y. M., Akewanlop, C., & Hayes, D. F. (1999). Antibody dependent cellular phagocytosis (ADCP) and antibody dependent cellular cytotoxicity (ADCC) of breast cancer cells mediated by bispecific antibody, MDX-210. Breast Cancer Res Treat, 53(3), 199-207.
Weiner, L. M., Murray, J. C., & Shuptrine, C. W. (2012). Antibody-based immunotherapy of cancer. Cell, 148(6), 1081-1084. doi: 10.1016/j.cell.2012.02.034
Weiner, L. M., Surana, R., & Wang, S. (2010). Monoclonal antibodies: versatile platforms for cancer immunotherapy. Nat Rev Immunol, 10(5), 317-327. doi: 10.1038/nri2744
Weiskopf, K., Ring, A. M., Ho, C. C., Volkmer, J. P., Levin, A. M., Volkmer, A. K., Garcia, K. C. (2013). Engineered SIRPalpha variants as immunotherapeutic adjuvants to anticancer antibodies. Science, 341(6141), 88-91. doi: 10.1126/science.1238856
Weiskopf, K., & Weissman, I. L. (2015). Macrophages are critical effectors of antibody therapies for cancer. MAbs, 7(2), 303-310. doi: 10.1080/19420862.2015.1011450

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method for increasing antibody-dependent cellular phagocytosis (ADCP) activity of a phagocytic cell in a sample comprising:
culturing the phagocytic cell in a low percent (%) CO₂ atmosphere for a period of time sufficient to increase ADCP activity of the phagocytic cell.

2. The method of claim 1, wherein the low % CO₂ is from about 0.1% CO₂ to about 1% CO₂.

3. The method of claim 2, wherein the low % CO₂ is about 1% CO₂.

4. The method of claim 1, wherein the period of time sufficient to increase ADCP activity of the phagocytic cells is 3 to 15 days.

5. The method of claim 1, further comprising determining the ADCP activity of the phagocytic cell using target cells labeled with a fluorescent dye.

6. The method of claim 1, wherein the phagocytic cell is a J774A.1 cell or a donor cell.

7. The method of claim 1, wherein the phagocytic cells are cultured in the presence of target cells.

8. The method of claim 7, wherein target cells are added to the phagocytic cell culture after about 7 days.

9. The method of claim 7, wherein the target cells are Daudi cells, B cells, leukemia cells or lymphoma cells.

10. The method of claim 5, wherein the fluorescent dye is pHrodo-Red, pHAb or AcidiFluor.

11. The method of claim 1, wherein expression of a regulator of ADCP is increased.

12. The method of claim 11, wherein the regulator is ATF4, FOXO3, IL1B, IL6, VEGFA, HGF, EGF, CHD1, SELP, TIMP3, DACH1, STAT3, GLI1, SP3, or combinations thereof.

13. The method of claim 1, wherein expression of a regulator of ADCP is decreased.

14. The method of claim 13, wherein the regulator is TP53, TNF, TGFB1, STAT6, MYD88, HRAS or combinations thereof.

15. The method of claim 7, wherein the target cells are contacted with an antibody or fragment thereof.

16. The method of claim 1, wherein the sample is cultured in a humidified chamber or incubator.

17. A method for assaying ADCP activity of a phagocytic cell in a sample comprising:
culturing the phagocytic cell in a low percent (%) CO₂ atmosphere for a period of time sufficient to increase ADCP activity of the phagocytic cell; and
detecting ADCP activity of the phagocytic cell in the sample.

18. The method of claim 17, wherein the low % CO₂ is from about 0.1 % CO₂ to about 1% CO₂.

19. The method of claim 18, wherein the low % CO₂ is about 1% CO₂.

20. The method of claim 17, wherein the period of time sufficient to increase ADCP activity of the phagocytic cells is 3 to 15 days.

21. The method of claim 17, further comprising determining the ADCP activity of the phagocytic cell using target cells labeled with a fluorescent dye.

22. The method of claim 17, wherein the phagocytic cell is a J774A.1 cell or a donor cell.

23. The method of claim 17, wherein the phagocytic cells are cultured in the presence of target cells.

24. The method of claim 23, wherein target cells are added to the phagocytic cell culture after about 7 days.

25. The method of claim 23, wherein the target cells are labeled with a fluorescent dye.

26. The method of claim 17, wherein the phagocytic cell is a J774A.1 cell or a donor cell.

27. The method of claim 23, wherein the target cells are Daudi cells, B cells, leukemia cells or lymphoma cells.

28. The method of claim 21, wherein the fluorescent dye is pHrodo-Red, pHAb or AcidiFluor.

29. The method of claim 17, wherein expression of a regulator of ADCP is increased.

30. The method of claim 29, wherein the regulator is ATF4, FOXO3, IL1B, IL6, VEGFA, HGF, EGF, CHD1, SELP, TIMP3, DACH1, STAT3, GLI1, SP3, or combinations thereof.

31. The method of claim 17, wherein expression of a regulator of ADCP is decreased.

32. The method of claim 31, wherein the regulator is TP53, TNF, TGFB1, STAT6, MYD88, HRAS or combinations thereof.

33. The method of claim 23, wherein the target cells are contacted with an antibody or fragment thereof.

34. The method of claim 17, wherein the sample is cultured in a humidified chamber or incubator.
